(19) United States
(12) Patent Application Publication
(10) Pub. No.: US 2022/0047317 A1
(43) Pub. Date: Feb. 17, 2022

(54) PERCUTANEOUS ULTRASOUND GUIDED MINIMALLY INVASIVE VEIN ABLATION METHOD FOR SAPHENOUS VEINS IN THE LOWER EXTREMITY

(71) Applicant: Ashanga Yatawatta, Bullhead City, AZ (US)

(72) Inventor: Ashanga Yatawatta, Bullhead City, AZ (US)

(21) Appl. No.: 16/947,677

(22) Filed: Aug. 12, 2020

(51) Int. Cl. A61B 18/06; A61M 25/10

(52) U.S. Cl. CPC A61B 18/06; A61B 17/122; A61M 25/10

(57) ABSTRACT

A vein ablation method is provided. The vein ablation procedure includes ultrasound guided cannulation of saphenous vein with a micro puncture method. An initially wire is placed across the vein to allow a vein stapler to be fired. Next the wire is place within the vein and after a sheath is placed, the wire-guided/venous catheter is placed into the vein as close to the foot as possible under ultrasound guidance. The ablation chemical is injected slowly while both the leg is elevated and while a compression stocking unrolled while the venous catheter is withdrawn. After removal of the sheath, placement of another vein stapler across the vein to isolate the segment of vein access site is an option.

### CROSS-REFERENCE TO RELATED APPLICATION

This application claims the benefit of priority of U.S. provisional application No. 62/841,883, filed 2 May 2019, the contents of which are herein incorporated by reference.

### BACKGROUND OF THE INVENTION

The present invention relates to surgical procedures for treating varicose veins and, more particularly, to a percutaneous ultrasound-guided minimally invasive vein ablation method for saphenous veins in the lower extremity.

Saphenous vein ablation is expensive and associated with significant risks yet struggles to treat the whole length of the vein. The existing methods typically use thermal energy that engenders the risk of thermal damage to surrounding structures as well as deep venous thrombosis. Moreover, the existing methods of saphenous vein ablation can treat only a limited part of the saphenous vein system.

Moreover, current methods for treating varicose veins between the knee to the groin utilize laser or radio frequency ablation. These approaches, however, need multiple injections of tumescent anesthetics along the whole pathway. As a result, pain and thermal damage to adjacent structures and deep vein thrombosis at the saphenofemoral junction are not uncommon. Furthermore, the access is typically antegrade and therefore the rest of the vein cannot be treated. In other words, a minimally invasive method to treat varicose veins present between the groin to the ankle as an office procedure does not exist today.

As can be seen, there is a need for a percutaneous ultrasound guided minimally invasive, retrograde vein ablation method for saphenous veins in the lower extremity.

The present invention embodies using ultrasound guidance with local anesthesia alone, wherein the percutaneous method may access the great saphenous vein at the saphenofemoral junction and retrogradely introduce a small catheter after wire access. An incision may be made at the groin with no other anesthetics needed. The venous catheter may be advanced up to the ankle level wherein injections of ablation chemical will be done. After completion of the injections, the wire may be used to introduce a stapling device to close off the vein. Prior to and immediately afterwards, compression stockings may be used after elevation of the leg to keep the veins collapsed for the ablation chemical to work.

The method embodied in the present invention is retrograde, allowing treatment of the whole length of the saphenous vein. The method also uses chemical ablation which becomes more physiological at the deep veins, minimizing the risk of deep vein thrombosis. Specifically, the present invention uses ablation chemical with no need for tumescent anesthesia nor thermal energy, and so the full length of the vein can be accesses via wire and catheter method. Thereby, decreasing the risks of deep vein thrombosis and eliminating the risk of thermal injury or pain. Furthermore, this method is cost effective, simple, and reliable.

### SUMMARY OF THE INVENTION

In one aspect of the present invention, a method of saphenous vein ablation, the method including retrogradely applying an ablation chemical from a distal-most portion of a saphenous vein to or adjacent to a first access site thereof at or near a saphenofemoral junction, whereby tumescent anesthesia or thermal energy is not incorporated in the method.

In another aspect of the present invention, method of saphenous vein ablation, the method including the following: over-the-wire (OTW) stapling a saphenous vein at a first access site of the saphenous vein at or near a saphenofemoral junction; OTW urging a venous catheter from a second access site to a distal-most portion of the saphenous vein, wherein the second access site is zero to three centimeters from the first access site; progressively urging an ablation chemical from a tip of said venous catheter during retrograde urging of said venous; and applying external compression to the saphenous vein during retrograde urging, whereby tumescent anesthesia or thermal energy is not incorporated in the method.

These and other features, aspects and advantages of the present invention will become better understood with reference to the following drawings, description and claims.

### BRIEF DESCRIPTION OF THE DRAWINGS

FIG. 1 is a cross section view of an exemplary embodiment of a vein staple insertion of the present invention;

FIG. 2 is a cross section view of an exemplary embodiment of the vein staple on the vein;

FIG. 3 is a cross section view of an exemplary embodiment of the vein staple attached;

FIG. 4 is a cross section view of an exemplary embodiment of a wire guided catheter of the present invention in an inserted condition;

FIG. 5 is a cross section view of an exemplary embodiment of a venous catheter of the present invention in an initial deployed state at a proximal end of the vein to be ablated;

FIG. 6 is a cross section view of an exemplary embodiment of the venous catheter in an expanding condition prior to urging toward a distal end of the vein to be ablated;

FIG. 7 is a cross section view of an exemplary embodiment of the venous catheter in the expanded condition in a pre-retrograde deployed state at or near the distal end of the vein to be ablated; and

FIG. 8 is a cross section view of an exemplary embodiment of the venous catheter in an expanded condition deployed in a post-retrograde deployed state prior to reversal of the inserted condition.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method of saphenous vein ablation through the retrograde over-the-wire catheter access and application of ablation chemical in such a way as to ablate the full length of the saphenous vein while obviating the need for tumescent anesthesia and thermal energy. The tip of the catheter can apply the ablation chemical to the distal-most part of the saphenous vein near the ankle and upward thereof as the catheter is withdrawn, while the leg is elevated and under externally applied compression.

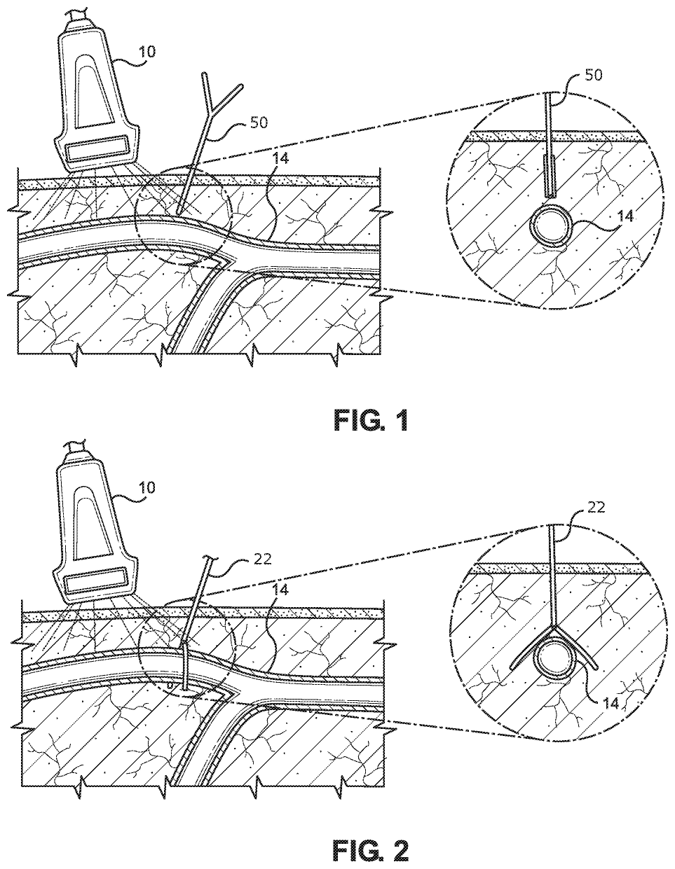

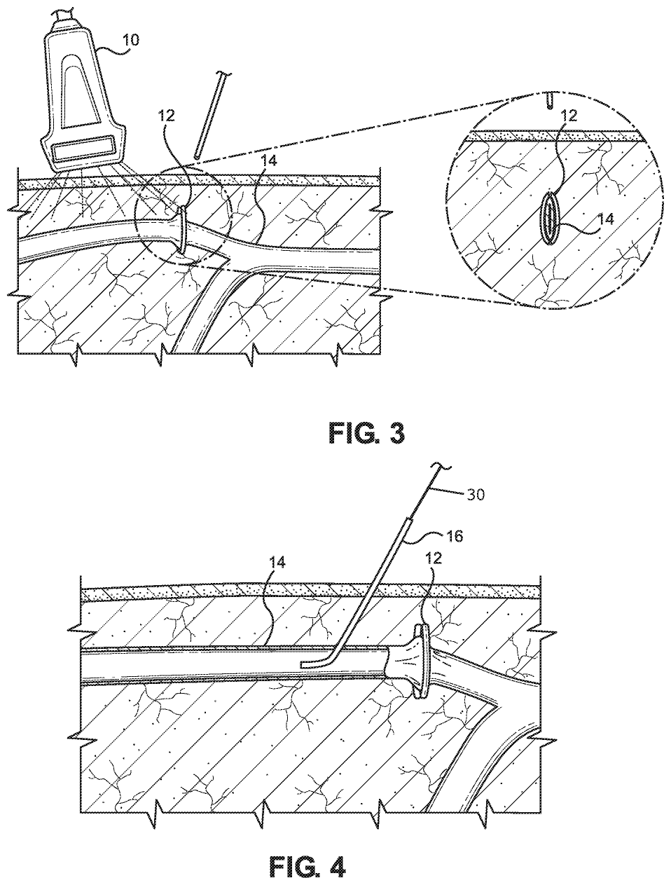

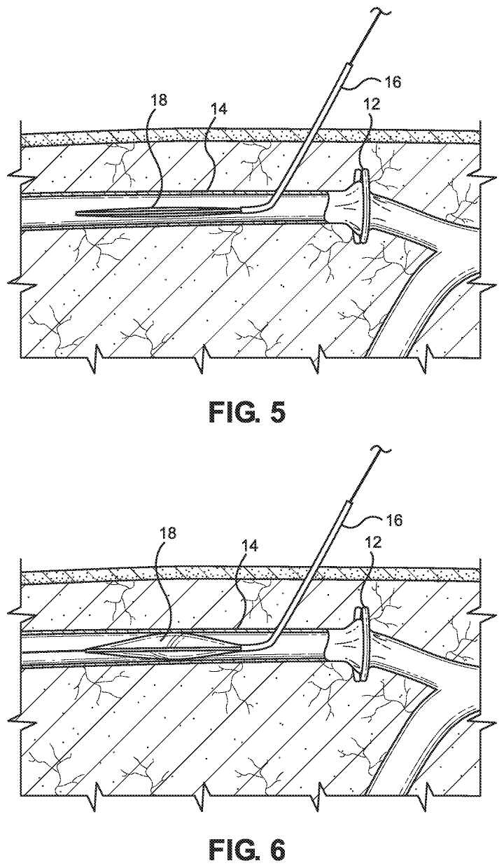

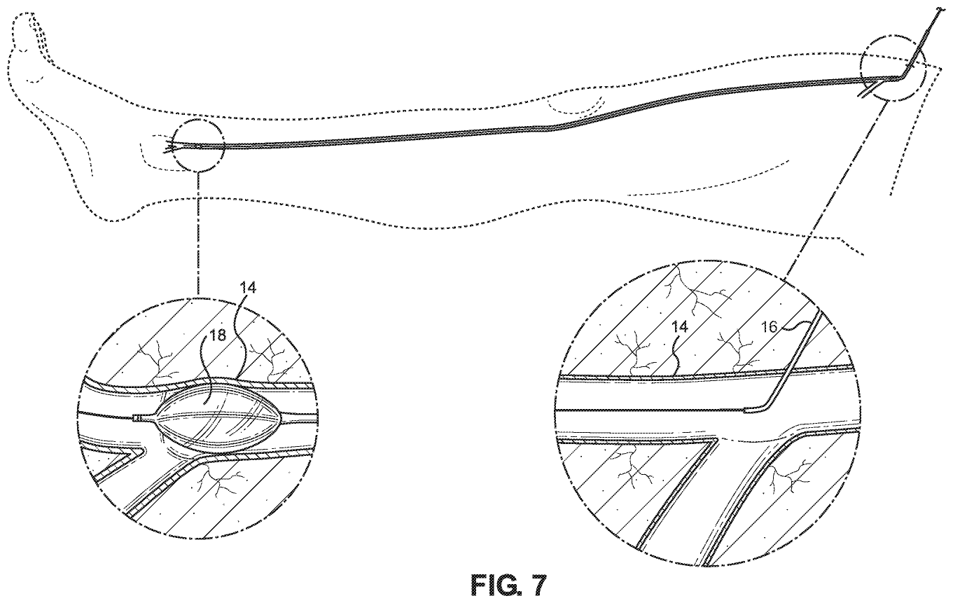

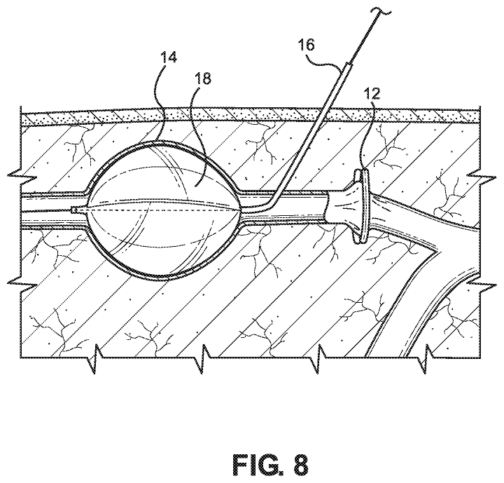

Referring now to FIGS. 1 through 8, the present invention may include a percutaneous ultrasound-guided minimally invasive vein ablation method for saphenous veins 14 in the lower extremity. The ablation method may embody a micro-catheter 16 with one or more side ports and a venous stapling device 22.

Through one of the ports of the micro-catheter assembly 16 and 18, a vein ablation chemical may be injected. The vein ablation chemical may be injectable irritant that is used in the treatment of varicose veins and that causes inflammation and subsequent fibrosis, thus obliterating the lumen of the vein; the ablation chemical may be but is not limited to sclerosant.

The micro-catheter assembly 16 and 18 and the venous stapling device 22 may be over-the-wire 30 (OTW), and so sequentially deployable and visible via diagnostic imaging techniques such as ultrasound. The micro-catheter assembly 16 and 18 with one or more side ports may be introduced OTW under ultrasound guidance to allow injection of ablation chemical.

The venous stapling device 22 may be introduced OTW (once ablation chemical has been injected) to deploy a venous staple 12 closing the saphenous vein 14 near the saphenofemoral junction. The vein stapler 22 may be similar to a closure device used in endovascular procedures. Each venous staple 12 may be a clip dimensioned and adapted to seal segments of the saphenous vein 14 of variable diameter. As a first step the venous stapling device 22 may be applied to the proximal end of the vein 14 near the saphenofemoral junction.

The micro-catheter assembly 16 and 18 may include a wire-guided catheter 16 and a venous catheter 18. The venous catheter 18 being dimensioned and adapted to be stiff yet flexible so as to be negotiated until the tip is in the distal-most part of the vein near the ankle. Once in place, the one or more side ports may be used to inject ablation chemical while the leg is elevated and under externally applied compression. The tip will be gradually withdrawn to the groin and wire access left in-situ while the venous catheter 18 is removed.

The venous catheter 18 may be made from vascular grade material based off similar models used for endovascular procedures or any suitable industry standard materials used for current endovascular catheters could be used to produce the venous catheter 18. The venous catheter 18 can be modified to offer an even less invasive approach even without an incision with smaller profile.

After completion of the retrograde application of the ablation chemical, the venous stapling device 22 may then be applied again just downward of the access site of the vein 14.

A method of using the present invention may include the following. The percutaneous, ultrasound-guided vein ablation method disclosed above may be provided. A medical professional, such as a surgeon, in an office setting with local anesthetics, may utilize a diagnostic imaging device 10 to assist in gaining access to the saphenous vein 14 in the groin region of a patient via an existing micro-puncture needle 50 for the vein stapling device 22 and the micro-catheter assembly: coaxial sheath/wire-guided catheter 16 and venous catheter 18. The vein stapling device 22 may be introduced so that the user may staple the vein 14 down near the saphenofemoral junction with clips.

The wire-guided catheter 16 may be introduced OTW to the vein 14 and wire-removed to push the venous catheter 18 down to the ankle. The patient's leg may be elevated and a compression stocking may be deployed at the same time as ablation chemical is being injected. The user may exchange the wire-guided catheter 16 via the wire 30, and then the vein stapling device 22 may be introduced so that the user may staple the vein 14 down near access site after the retrograde application of the ablation chemical. The vein stapler 22 could also be used for other percutaneous-based closure of blood vessels.

In certain embodiments, the following components of the vein ablation method may be as followings. First, the ultrasound guided micro puncture method is used to place a wire 30 across the vein 14 just distal to the saphenofemoral junction, at a first access site. Second, the vein stapler 22 is deployed to close off the first part of the saphenous vein 14 at the access site. Third, ultrasound guided micro puncture method used to place the wire 30 at a second access site, within the lumen of the saphenous vein 14, just distal of the first access site followed by the sheath/wire-guided catheter 16 and the venous catheter 18, whereby the expandable venous catheter 18 may be urged to a distal end of the saphenous vein 14. Fourth, ablation chemical may be retrogradely introduced via the venous catheter 18 under ultrasound guidance while the elevated limb is externally compressed with a compression stocking while slowly withdrawing the venous catheter 18. Fifth, after completion of the injection, another vein staple 12 may be applied in a third access site, isolating the vein segment with the second access site between two stapled portions (first and third access sites), each stapled portion approximately zero to three centimeters from the second access site.

Additionally, the percutaneous vein ablation method disclosed above can be used for any site with varicose veins including the short saphenous system.

It should be understood, of course, that the foregoing relates to exemplary embodiments of the invention and that modifications may be made without departing from the spirit and scope of the invention as set forth in the following claims.

What is claimed is:

1. A method of saphenous vein ablation, the method comprising:
retrogradely applying an ablation chemical from a distal-most portion of a saphenous vein to or adjacent to a first access site thereof at or near a saphenofemoral junction,
whereby tumescent anesthesia or thermal energy is not incorporated in the method.

2. The method of claim 1, the method further comprising:
initially stapling the saphenous vein at the first access site.

3. The method of claim 2, wherein the stapling is done over-the-wire.

4. The method of claim 3, prior to retrograde application of the ablation chemical, urging an expandable catheter through the saphenous vein from a second access site to said distal-most portion, the second access site distal of the first access site zero to three centimeters.

5. The method of claim 4, wherein the urging is done over-the-wire.

6. The method of claim 5, wherein the ablation chemical is urged through a tip of the expandable catheter.

7. The method of claim 6, wherein the ablation chemical is progressively urged through said tip moving from said distal-most portion to or adjacent to said first access site.

8. The method of claim 5, wherein the ablation chemical is sclerosant.

9. The method of claim 5, the method further comprising:
applying external compression to the saphenous vein during application of the ablation chemical.

10. The method of claim 9, the method further comprising:
stapling a third access site of the saphenous vein, the third access site distal of the second access site by zero to three centimeters.

11. A method of saphenous vein ablation, the method comprising:
over-the-wire (OTW) stapling a saphenous vein at a first access site of the saphenous vein at or near a saphenofemoral junction;
OTW urging a venous catheter from a second access site to a distal-most portion of the saphenous vein, wherein the second access site is zero to three centimeters from the first access site;
progressively urging an ablation chemical from a tip of said venous catheter during retrograde urging of said venous; and
applying external compression to the saphenous vein during retrograde urging,
whereby tumescent anesthesia or thermal energy is not incorporated in the method.

12. The method of claim 11, wherein the ablation chemical is sclerosant.

13. The method of claim 11, the method further comprising:
stapling a third access site of the saphenous vein, post retrograde urging, wherein the third access site distal of the second access site by zero to three centimeters.
